(19) United States
(12) Patent Application Publication
(10) Pub. No.: US 2008/0317794 A1
(43) Pub. Date: Dec. 25, 2008

(54) Agglomerate Particles, Method for Producing Nanocomposites, and the Use Thereof

(75) Inventors: Carsten Gellermann, Gerbrunn (DE); Herbert Wolter, Tauberbischofheim (DE); Werner Storch, Hochberg (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: FRAUNHOFER-GESELLSCHAFT ZUR FORDERUNG DER ANGEWANDTEN FORSCHUNG E.V., MUNCHEN (DE)

(21) Appl. No.: 12/097,873

(22) PCT Filed: Dec. 22, 2006
(86) PCT No.: PCT/EP06/12509

§ 371 Date: Aug. 28, 2008

(30) Foreign Application Priority Data
Dec. 23, 2005 (DE) 10 2005 061 965.7

(51) Int. Cl. A61K 9/14; B32B 15/16; B32B 27/00; B32B 9/00

(52) U.S. Cl. 424/401; 428/402; 428/405; 428/407; 524/606; 524/589; 524/599; 524/566; 524/571; 524/577; 524/584; 524/586; 524/560; 524/588; 524/609; 252/519.33; 424/489

(57) ABSTRACT

The invention relates to agglomerate particles which are constructed from agglomerated nanoscale primary particles and also to a method for incorporation of these particles in a polymer matrix and to the use of these agglomerate particles.

The invention relates to agglomerate particles which are constructed from agglomerated nanoscale primary particles and also to a method for incorporation of these particles in a polymer matrix and to the use of these agglomerate particles.

The production of nanoparticles (<1 μm primary particle size) is known in the literature and is achieved via various different methods. Thus methods in the gas phase (pyrolysis, PVD, CVD, etc.) enable the synthesis of more or less agglomerated or aggregated particles as powder. It is unfavourable thereby that for low agglomeration and aggregation degrees which are difficult to adjust, the synthesis and processing is all the more complex and cost-intensive. Surface modifications on these particles, such as for example, hydrophobing, modifying with active substances or functionalising for covalent bonding of the particles into a polymer matrix are likewise often very complex and only possible subsequently and lead generally to agglomerates and aggregates.

In order to shield the nanoscale primary particles mutually, frequently large quantities of additives, dispersion aids etc. are used. Nevertheless, an agglomeration/aggregation of the particles cannot be avoided completely in this way. For many applications, these additives are in addition disruptive. Further disadvantages can be seen for example with respect to the processing in the much too low densities (bulk density, compacted bulk density etc.) (cf. e.g. Aerosil by Degussa). Isolation of detached nanoparticles is therefore difficult.

In order to circumvent problems of particle isolation whilst avoiding agglomeration/aggregation, oxidic nanoparticles are frequently produced by the wet-chemical route, are silanised in situ and stabilised in dispersion. The thus produced dispersions containing up to 50% by weight solid content can be hydrosols or organosols. The large quantity of (reactive) solvents requiring to be used, which are difficult to remove and hence regarded in general as an encumbrance, is disadvantageous with the organosols. These must be process friendly and—according to the application—compatible with a specific polymer. In addition, a particle product based on powder is generally preferred from the point of view of the user for process-technological reasons.

Starting herefrom, it is therefore the object of the present invention to indicate particles which enable problem-free further processing into products containing nanoparticles and also to indicate a method for the production of composites of this type.

The object is achieved with respect to the particles by the features of claim 1, with respect to the method for the production of polymer matrices containing particles by the features of claim 10. The sub-claims reveal advantageous developments.

According to claim 1, agglomerate particles in solid form which are constructed from nanoscale primary particles are proposed. The core of the teaching according to the invention resides in the fact that, for processing nanoparticles, it is not the nanoparticles which are made available as such in the form of a wet-chemical dispersion but rather that the nanoparticles are present in the form of agglomerated but also specifically comminuted nanoscale primary particles. These thus agglomerated nanoscale primary particles then form the agglomerate particles according to the invention with an average particle diameter of 0.5 to 100 μm, preferably with a particle diameter of 1 to 20 μm.

The agglomerate particles according to the invention combine the advantageous properties of microparticles with those of nanoparticles. Because of the procedural agglomeration of primary nanoparticles into agglomerate particles of micrometre size, the properties thereof correspond to those of standard microparticles. The lower tendency to dust which is important for processing, the good flowability thereof and the health aspect in comparison with nanoparticles (low lung exposure) are advantageous. This a substantial advantage for the safety at work of the workers handling powders. It should be emphasised in this context above all also that the novel agglomerate particles, in contrast to other nanopowders, can be processed for the first time without great additional complexity in a conventional manner, i.e. in normal machines, such as laboratory extruders.

A further advantage of the invention resides in the minimisation of the surface energy of the particles due to the combination of different effects:
geometry
chemical modification
adaptation of the particle surface to the matrix (e.g. good wettability).

The contact faces of the primary particles have been minimised successfully, i.e. at most a point-wise contact can be set relative to each other. This is successful in particular when the primary particles are approximately spherical.

The primary particles are preferably selected thereby such that the formed agglomerate particle disintegrates again into the nanoparticles during incorporation, e.g. under the effect of shear forces. In this way, nanoparticles can hence be incorporated in a polymer without difficulty even up to high filling degrees, namely up to 40% by weight.

The agglomerate particles according to the invention are characterised by their uniform shape (e.g. spherical or doughnut-like, hollow or compact), good flow behaviour, low swelling effect, simple processibility and adjustable ability to be comminuted, i.e. for example a high ability to be deagglomerated with low energy expenditure.

In the case of nanoscale primary particles, it is favourable if these have essentially the same particle size so that approximately uniform, preferably spherical agglomerate particles are then also produced in the end effect. The primary particles can thereby be present also in a core-shell form or as hollow balls. However it is also likewise possible that three-dimensional, two-dimensional or one-dimensional nanosystems, e.g. balls, all crystal structures, small plates, layer silicates and other layer systems, fibres, tubes, wires etc., are used.

In the case of the primary particles, in particular oxidic ones are preferred which are selected preferably from the following metals: silicon, tin, titanium, zirconium, calcium, strontium, barium, aluminium, yttrium, zinc, tantalum, cerium, gadolinium, holmium, erbium, ytterbium and also glass, glass ceramics and ceramic-forming materials and/or combinations thereof. The material can be both crystalline, partially crystalline and also amorphous. Likewise, it is possible that the nanoscale primary particles comprise further metal compounds, e.g. metal phosphates, metal sulphates, metal sulphites, metal sulphides, metal selenides, metal carbonates, metal nitrates, metal borates, metal carbides, metal halogens or mixtures thereof or contain these. Furthermore, it is preferred that the primary particles comprise pure metals.

The agglomerate particles can be combined arbitrarily with other fillers and/or additives for the production of composites or nanocomposites. In the case of the fillers, these can be any particles, fibres in the nano- or micrometre range or fabric. Examples of these are classic glass fillers, glass fibres and fabrics, silicate fillers, polysilicic acids, metals and metal oxides, polymers (e.g. beads), carbon (graphite, carbon black, fullerene, C-nanotubes, nano- and microfibers), barium sulphate, silicon carbide, silicon nitride, titanates and aluminium hydroxide (ATH). In the case of the additives, these can be different normal stabilisers (inhibitors, antioxidants, biostabilisers, light protection agents (UV absorbers) or metal deactivators), processing aids, (lubricants, separating means, slip-, antislip-, anti-blocking agents etc.), mechanical additives (plasticisers, impact strength modifiers etc.) and also product modifiers (antistatics, fire retardants, nucleus formers for partially crystalline polymers, propellants, optical lighteners, pigments/colourants etc.).

It is preferred with the agglomerate particles according to the invention if these are constructed from nanoscale primary particles, the surfaces of which have been functionalised. Agglomerate particles of this type, i.e. agglomerate particles which were formed from surface-modified nanoparticles, display excellent deagglomeration when they are incorporated in a polymer matrix for example under the effect of shear forces. It is therefore preferred in the invention that, in the application cases where deagglomeration of the agglomerate particles is sought, those are used which are constructed from surface-modified primary particles.

Furthermore, chemical modification for setting defined interparticulate interactions is advantageous. These interactions must be expressed in such a manner that the primary particles, on the one hand, are held together loosely at points and, on the other hand, can be comminuted if required without great expenditure (shear forces). It is thereby essential to minimise the OH groups present on the surface—in the case of SiO₂ these are for example silanol groups—in order that an interparticulate irreversible condensation and hence an aggregate formation is avoided. The adaptation of the particle surface to the polymer matrix should be mentioned as a further essential aspect.

According to the invention, both nanoparticle-containing aqueous hydrosols and organosols can serve as starting basis. These can be produced via known methods. Nanoparticles comprising for example SiO₂, TiO₂, SnO₂, ZrO₂ etc. have proved to be successful for the production of comminutable agglomerate particles. The average particles sizes are thereby in the range of 2 to 500 nm. Particles with narrow particle size distributions are preferably used.

Also commercial sols, such as for example a silica sol or an aqueous ZrO₂ sol, can be used. Pure water or also hydrophilic, hydrophobic, protonic or aprotonic organic solvents can thereby serve as solvents. Amongst the organic solvents, alcohols or ketones are preferred. Mono-, bi- and trivalent alcohols are suitable. As trivalent alcohol there is suitable for example glycerine and, as bivalent alcohols, there are suitable e.g. ethylene glycol or the propane diols. Particularly suitable are the monovalent alcohols, in particular those with up to five C-atoms in the molecule. Mixtures of water with organic compounds are preferred. In particular there are preferred acetone, tert.-butylalcohol, methanol, ethanol and also n- and i-propanol.

A surface modification of the primary particles which is required with respect to the ability to be comminuted of the agglomerate particles to be produced is achieved by a combination of the described sols with special chemical substances, such as silanes, silanzanes, siloxanes, carboxylic acids, alcohols, amines, amino acids, sulphonates, (poly)phosphates, ampholytes, thermoplastic homopolymers, (block)copolymers and/or combinations thereof. Silanes have proved to be particularly advantageous for oxidic primary particles. These differ in the type and number of hydrolysable groups thereof. Preferred silanes are alkoxysilanes, in particular mono-, di-, trialkoxy- or -chlorosilanes, such as trimethylchlorosilane, trimethylmethoxysilane, dimethyldichlorosilane, dimethyldimethoxysilane, 3-aminopropyltrimethoxysilane, 3-aminopropyltriethoxysilane, 3-chloropropyltrimethoxysilane, 3-chloropropyltriethoxysilane, 2-aminoethyl-3-amino-propyl-trimethoxysilane, 3-glycidyloxypropyltrimethoxysilane, 3-glycidyloxypropyltriethoxysilane, hexadecyltrimethoxysilane, isobutyltrimethoxysilane, isobutyltriethoxysilane, 3-mercaptopropyltrimethoxysilane, 3-methacryloxypropyltrimethoxysilane, 3-methacryloxypropyltriethoxysilane, methyltrimethoxysilane, methyltriethoxysilane, octyltrimethoxysilane, octyltriethoxysilane, pentyltrimethoxysilane, propyltrimethoxysilane, propyltriethoxysilane, (2-aminoethyl)-2-aminoethyl-3-amino-propyltrimethoxysilane, vinyltrimethoxysilane, vinyltriethoxysilane, tetramethoxysilane, tetraethoxysilane.

Particularly good effects with respect to the ability to be comminuted of the agglomerate particles by means of specific combinations of individual chemical substances are thereby frequently made possible. The combination of a silane with a different chemical substance for example has proved to be favourable.

Preferably, the silanes have an organofunctional group which are available after surface modification for conversion or reaction with corresponding partners, such as the polymer matrix. Such functional groups are for example acryl or methacryl, vinyl, amino, mercapto, substituted carboxy groups, alkyl groups, in particular C1- to C20-alkyl groups, alkoxy groups, cyano groups, isocyano groups, cyanato groups, isocyanato groups, acid anhydrides, in particular cyclic anhydrides, such as succinic anhydride, epoxy groups, such as in particular ethoxy groups, such as glycidoxy groups, carbamates etc. Reaction partners of this type make possible for the first time optimum adaptation of the particle surfaces to the polymer matrix and also meaningful covalent binding with respect to the mechanical properties of the resulting nanocomposites.

The chemical substances which can be used for the surface modification are applied under special synthesis conditions to the particle surfaces of the primary particles with condensation. A subsequent condensation thereby takes place during the drying process. Essential parameters in the surface modification are above all kind and type of reactands, reaction medium, the pH value and the temperature and also the drying conditions used during the powder isolation.

In general, the resulting agglomerate particles are of an oxidic nature and essentially contain oxygen. Instead of or in addition to the oxygen, also other elements can be contained in the anions, such as for example hydrogen (e.g. as hydroxide), sulphur (e.g. sulphide, sulphite, sulphate), phosphorus (e.g. phosphate), halogens (e.g. perchlorate, chlorate). They can also contain glass, glass ceramic and ceramic-forming materials. The material can be both crystalline, partially crystalline and amorphous. The morphology of the agglomerate particles, with respect to the ability to be comminuted, is not relevant and therefore not critical. Since these particles are constructed from primary particles they contain therebetween cavities (gussets) which are responsible for a defined porosity. This is independent of the primary particle size but rather depends much more upon the surface energy and hence also upon the interactions between the particles.

After comminution in the machines known from the plastic material industry (e.g. extruders), nanocomposites are obtained under the effect of shear forces, ultimately the advantageous properties of the nanocomposites produced in a complex manner via other methods being achieved.

Tests on the ability to be comminuted were implemented in aqueous and organic solvents. For this purpose, a defined quantity of agglomerate particles was added to e.g. ethanol and subjected to an ultrasound treatment over a period of up to 30 min. The energy supplied by this treatment is relatively low in comparison with the strong shear forces produced in the laboratory extruder with twin-screws and the thus associated high total energy supply. Therefore, the agglomerate particles which can already be comminuted rapidly in the laboratory by means of ultrasound should likewise be able to be comminuted well in the extruder.

The invention relates in addition to a method for the incorporation of agglomerate particles, as described above, in a polymer matrix. It should be emphasised hereby in particular that it is possible with the agglomerate particles according to the invention to incorporate up to 40% by weight relative to the total mass of the polymer matrix. Incorporation of the agglomerate particles in the polymer matrix can thereby by effected on agglomerate particles which are present in solid form in such a manner that these are introduced directly into a corresponding device, e.g. into an extruder, or else in that the agglomerate particles are present in a master batch and this master batch is then combined with the polymer matrix according to known methods.

The specific/controlled agglomeration of the primary nanoparticles into the agglomerate particles can be effected according to the present invention according to methods known per se, such as spray drying, spray freeze drying, freeze drying and/or aerosol methods.

If the agglomerate particles are provided for processing during which they are intended to disintegrate into nanoparticles, it has proved to be favourable if then the agglomerate particles are constructed from surface-functionalised primary particles, silane-modified primary particles being used here particularly preferably. All the methods in which the nanoparticles are incorporated in the polymer matrix by means of shear forces have proved in particular to be suitable methods for the incorporation. In this case the result is then disintegration of the agglomerate particles into nanoparticles. Of course, it is hereby possible that in addition to the effect of shear forces also a separate ultrasound treatment is implemented.

After their incorporation in the polymer, the agglomerate particles, during comminution into smaller agglomerate particles under shearing for example in the laboratory extruder (sub-units), effect a low to high viscosity increase in the polymer according to the particle content and surface-matrix adaption. In the case of a matrix-adapted particle surface, only a low viscosity increase was observed even at high contents up to approx 40% by weight. Intermediately, the result can however be an increase in viscosity which has proved to be advantageous with respect to comminution of the agglomerates.

The resulting nanocomposites (e.g. made of PMMA) are characterised by nanoparticle-related increases for example in the modulus of elasticity and scratch resistance with essentially unchanged transparency and impact strength. Further properties which as a rule are advantageously changed by the particles relate for example to the hardness, friction behaviour, stress-cracking resistance, rigidity, strength, thermal properties, such as length expansion, burning behaviour, heat conductivity; resistance to environmental influences, such as for example to light and temperature change; gas and water vapour permeability and also optical properties such as gloss.

In addition to the above-described incorporation of the agglomerate particles in a polymer matrix, it has been shown that the agglomerate particles according to the invention are also suitable for many further applications. In general, the agglomerate particles according to the invention are suitable for the production of nanodispersions, nanocomposites and/or nanocoatings. A preferred application is the production of varnishes, paints, inks, coating systems, flame protection systems and/or electrorheological liquids. The agglomerate particles are also suitable for the production of composites for dielectric, electrooptical and electrical applications and also for medical applications and as cosmetic, pharmaceutical or medical aids.

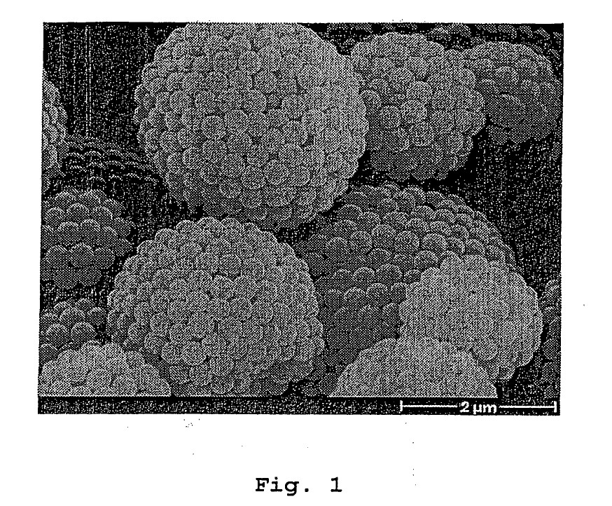

The invention is described subsequently in more detail with reference to embodiments and to FIGS. 1 to 10.

FIG. 1 shows the construction of an agglomerate particle according to the invention

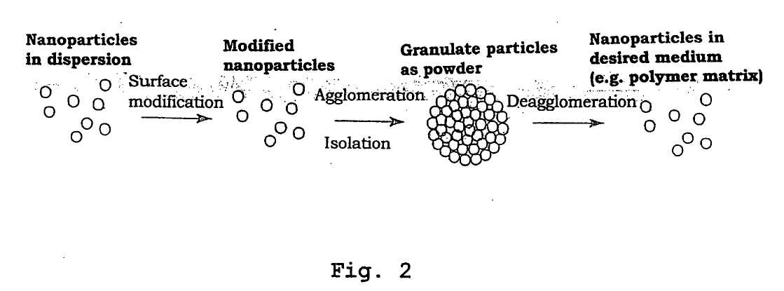

FIG. 2 shows a basic diagram of the agglomerate formation and deagglomeration

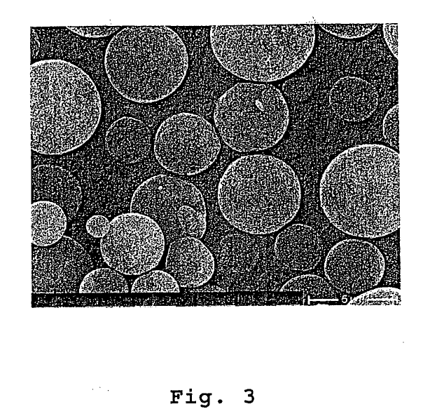

FIGS. 3 to 5 show SEM images of agglomerate particles according to the invention

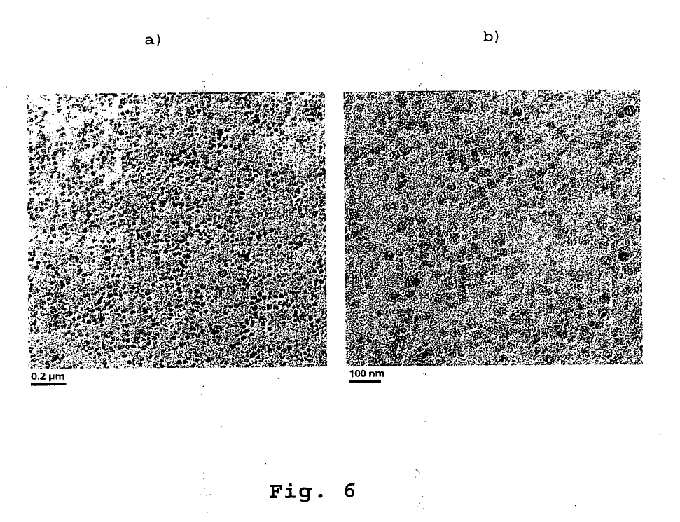

FIGS. 6a and 6b thereby show TEM images of a 20% by weight particle-containing PMMA test bar

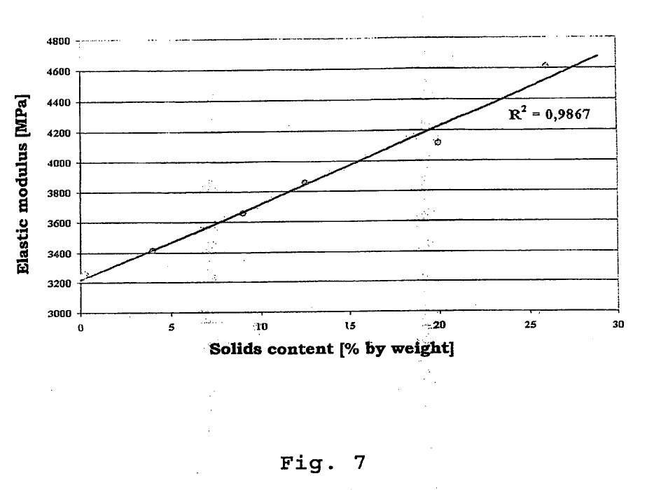

FIG. 7 shows the behaviour of the modulus of elasticity of a PMMA test bar as a function of filling level with agglomerate particle

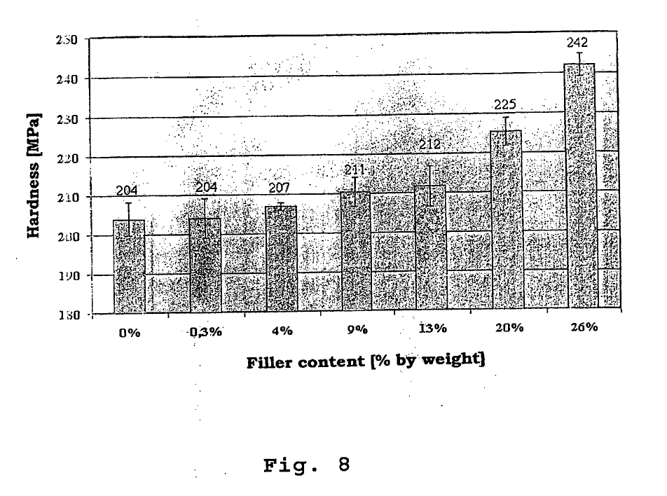

FIG. 8 shows the corresponding change in Vickers hardness

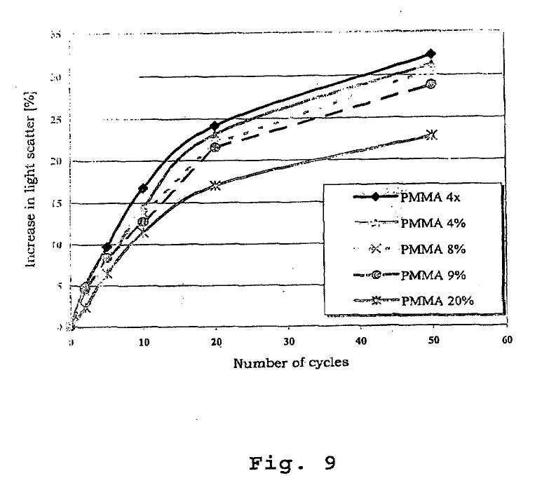

FIG. 9 shows the test results for abrasion by means of Taber-abraser method

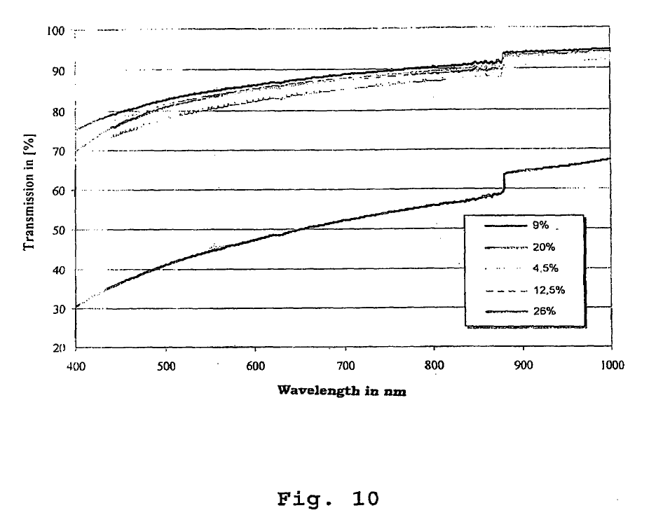

FIG. 10 shows UV/VIS measurements in the wavelength range of 400 to 100 nm of a PMMA disc filled up to 20% by weight.

FIG. 1 now shows the construction according to the invention of an agglomerate particle. The agglomerate particle according to FIG. 1 is constructed from a large number of individual primary particles. SiO₂ particles were used as primary particles. The average particle diameter is 3 μm.

FIG. 2 now shows the basic diagram for modification, agglomeration and isolation of dispersed nanoparticles in the form of agglomerate particles and the deagglomeration in a dispersion agent.

The invention is explained subsequently in more detail by means of examples.

### 1. Spherical Agglomerate Based on SiO₂

To 100 ml of a commercial 40% by weight silica sol (Ludox, co. DuPont), g Aerosil 380 and if necessary alcohol are added slowly and with vigorous agitation. If necessary, this is diluted with 50 ml water in order to adjust a sprayable sol. Subsequently, spray drying with a mini spray dryer model 190 of the co. Büchi is implemented. The following settings were thereby chosen:
two material nozzle diameter: 0.5 μm
atomiser medium: air
input temperature: 180° C.
output temperature: 76° C.
pump power: 70%
aspirator setting: 100%

The result was 46 g of a white powder with an average particle size of 3 μm, measured by means of Fraunhofer diffraction. The particles are relatively compact but have a certain porosity (no hollow particles) because of the particle-based synthesis. The test for deagglomeration (dispersion in ethanol by means of ultrasound and subsequent measuring by means of dynamic light scatter) produced an average particle size of 2.4 μm (FIG. 3).

### 2. Spherical Agglomerate Based on SiO₂ and SnO₂

50 g of a commercial 40% by weight silica sol (Ludox, co. DuPont) and 133% by weight of an SnO₂ dispersion (15% by weight SnO₂, co. Alfa Aesar, containing SnO₂ particles of 15 nm size) were combined with vigorous agitation. If necessary this was diluted with 50 ml water in order to adjust a sprayable sol. Subsequently, spray drying with a mini spray dryer model 190 of co. Büchi was implemented. The following settings were thereby chosen:
two material nozzle diameter: 0.5 μm
atomiser medium: air
input temperature: 180° C.
output temperature: 88° C.
pump power: 30%
aspirator setting: 100%

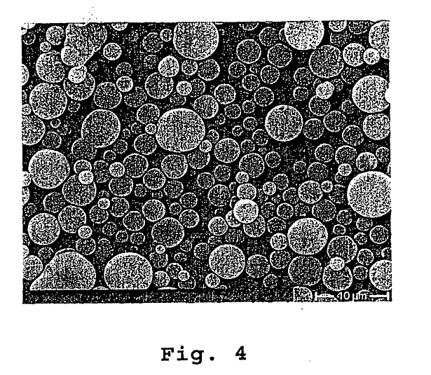

The result was 31 g of a white powder with an average particle size of 4 μm, measured by means of Fraunhofer diffraction. The x-ray fluorescence analysis produced an SnO₂ content of 47% by weight. The test for deagglomeration (dispersion in ethanol by means of ultrasound and subsequent measuring by means of dynamic light scatter) ran negatively, produced an average particle size of 2.6 μm (FIG. 4).

### 3. Annular Agglomerate Based on SiO₂

10 ml of a commercial 40% by weight silica sol (Ludox, co. DuPont) were mixed with 90 ml water. Subsequently, spray drying with a mini spray dryer model 190 of co. Büchi was implemented. The following settings were thereby chosen:
two material nozzle diameter: 0.5 μm
atomiser medium: air
input temperature: 220° C.
output temperature: 136° C.
pump power: 10%
aspirator setting: 100%

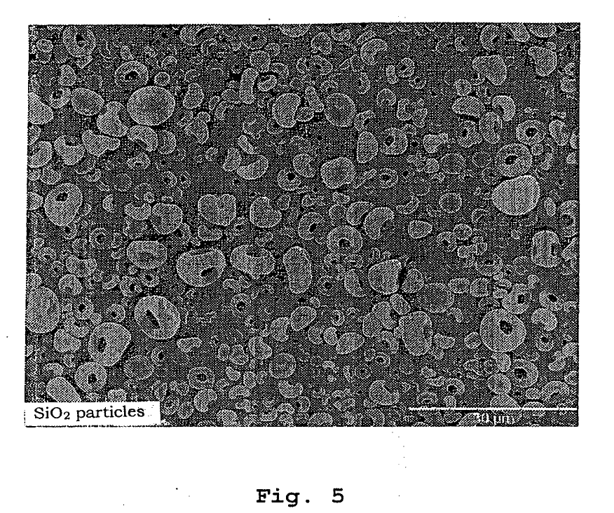

The result was 2 g of a white powder with an average particle size of 7 μm, measured by means of Fraunhofer diffraction. The test for deagglomeration (dispersion in ethanol by means of ultrasound and subsequent measuring by means of dynamic light scatter) ran negatively, produced an average particle size of 5.8 μm (FIG. 5).

### 4. Agglomerate Comprising Methacryl-Modified SiO₂

125 g of a commercial silica sol (Köstrosol 2040, co. Chemiewerk Bad Köstritz containing SiO₂ particles of 25 nm size) were mixed with 45 g water. Furthermore, 5.8 g (3-methacryloxy)propyltrimethoxysilane and if necessary alcohol were added slowly and refluxed with constant agitation over of period of 6 h (oil bath temperature: 120° C.). Since the resulting batch is very viscous, it is diluted with approx. 100 ml water in order to adjust a sprayable sol. Subsequently, spray drying with a mini spray dryer model B-191 of co. Büchi is implemented. The following settings were thereby chosen:
two material nozzle diameter: 0.5 μm
atomiser medium: air
input temperature: 130° C.
output temperature: 80° C.
pump power: 2
aspirator setting: 2

The result was 37 g of a white powder with an average particle size of 6 μm, measured by means of Fraunhofer diffraction. The test for deagglomeration (dispersion in ethanol by means of ultrasound and subsequent measuring by means of dynamic light scatter) ran positively and produced an average particle size of 40 nm. Detection of any methacryl groups present was effected with the help of diffuse reflection FTIR spectroscopy (DRIFT) by means of signals of the C═O stretch vibrations between 1700 and 1750 cm⁻¹.

### 5. Agglomerate Comprising Methacryl-Modified SnO₂

70 g of a commercial SnO₂ dispersion (15% by weight SnO₂ in H₂O, co. Alfa Aesar containing SnO₂ particles of 15 nm size) were mixed with 30 g water. Furthermore, 3.2 g (3-methacryloxy)propyltrimethoxysilane and 200 ml methanol were added slowly and refluxed with constant agitation over a period of 6 h (oil bath temperature: 120° C.). Since the resulting batch is very viscous, it is diluted if necessary with approx. 50 ml water in order to adjust a sprayable sol. Subsequently, spray drying with a mini spray dryer model B-191 of co. Büchi was implemented. The following settings were thereby chosen:
two material nozzle diameter: 0.5 μm
atomiser medium: air
input temperature: 130° C.
output temperature: 80° C.
pump power: 2
aspirator setting: 2

The result was approx. 8 g of a white powder with an average particle size of 5 μm, measured by means of Fraunhofer diffraction. The test for deagglomeration (dispersion in solvent by means of ultrasound and subsequent measuring by means of dynamic light scatter) ran positively and produced an average particle size of 30 nm. Detection of any methacryl groups present was effected with the help of diffuse reflection FTIR spectroscopy (DRIFT) by means of signals of the C═O stretch vibrations between 1700 and 1750 cm⁻¹.

### 6. Composite-Agglomerate Comprising SiO₂ and Polyacrylic Acid

125 g of a commercial silica sol (Köstrosol 2040, co. Chemiewerk Bad Köstritz containing SiO₂ particles of 25 nm size) were mixed with 45 g water. Furthermore, 20 g polyacrylic acid were added slowly and refluxed with constant agitation over a period of 6 h (oil bath temperature: 120° C.). If necessary, it was diluted with approx. 100 ml water in order to adjust a sprayable sol. Subsequently, spray drying with a mini spray dryer model B-191 of co. Büchi was implemented. The following settings were thereby chosen:
two material nozzle diameter: 0.5 μm
atomiser medium: air
input temperature: 130° C.
output temperature: 80° C.
pump power: 2
aspirator setting: 2

The result was 25 g of a white powder with an average particle size of 8 μm, measured by means of Fraunhofer diffraction. The test for deagglomeration (dispersion in ethanol by means of ultrasound and subsequent measuring by means of dynamic light scatter) produced an average particle size of 640 nm. Detection of any methacryl groups present was effected with the help of diffuse reflection FTIR spectroscopy (DRIFT) by means of intensive signals of the C═O stretch vibrations between 1700 and 1750 cm⁻¹.

### Examples for Incorporation of Agglomerate Particles in Polymethylmethacrylate (PMMA)

### COMPOSITE EXAMPLE

Agglomerate particles made of methacryl-modified SiO₂ (see example 4) were incorporated with a twin-screw extruder (model ZK 25T, co. Collin) in different conditions up to 26% by weight in thermoplastic polymethylmethacrylate (PMMA 8N, co. Röhm). Firstly, a master batch was produced which was used later for dilution with PMMA. For the master batch production, the material was extruded three times, i.e. respectively once for incorporation, homogenising and degassing, through the small processing machine. A fourth extrusion was effected in order to adjust the different contents by dilution with pure PMMA. For the characterisation, both test bars (mechanics), foils (abrasion) and discs (transparency) were produced. The TEM images, shown in FIGS. 6a and b, of the resulting PMMA-based nanocomposites containing 20% by weight SiO₂ show impressively that the shear forces of the twin-screw extruder obviously suffice to disintegrate agglomerate particles produced by means of spray drying into the individual components (primary particles).

The properties of the PMMA can be increased by incorporation of methacryl-modified SiO₂ particles in the form of agglomerate particles, as the following examples show.

Filing of PMMA with up to 26% by weight agglomerate particles causes an increase in the modulus of elasticity by up to 43% (see FIG. 7).

At the same time, an increase by 19% is revealed in the Vickers hardness (see FIG. 8).

Tests for abrasion by means of Taber-abraser method (50 cycles) confirm that the filling of PMMA with 20% by weight particles causes a reduction in the light scatter by 30% (see FIG. 9).

UV/VIS measurements in the wavelength range of 400 to 1000 nm show the comparatively high transmission of the PMMA discs filled up to 20% by weight and hence confirm the high deagglomeration degree of the granulate powder (see FIG. 10).

Characterisation with Respect to Deagglomeration

In order to determine the deagglomeration degree, respectively 0.5 g of the resulting powder (agglomerate particles) in 10 g of a solvent (water or ethanol) were added and treated by means of ultrasound. An ultrasound bar was used for this purpose which was immersed directly in the dispersion. A time-dependent size determination of the granulates was implemented by means of Fraunhofer diffraction and dynamic light scatter (photon correlation spectroscopy). For this purpose, a small component of the dispersion was removed after defined time intervals (10, 20 and 30 min.). It emerged that, respectively according to surface modification of the nanoparticles used and as a function of the production process, a deagglomeration can be adjusted. This means that average diameters of below 100 nm were measured in individual samples.

1. Agglomerate particles having an average particle diameter of 0.5 to 100 μm comprising agglomerated nanoscale primary particles having an average particle size in the range of 2 to 500 nm.

2. Agglomerate particles according to claim 1, wherein the agglomerate particles are spherical.

3. Agglomerate particles according to claim 1, wherein the nanoscale primary particles are spherical.

4. Agglomerate particles according to claim 1, wherein the nanoscale primary particles essentially have the same particle size.

5. Agglomerate particles according to claim 1, wherein the average particle diameter of the agglomerate particles is in the range of 1 to 20 μm.

6. Agglomerate particles according to claim 1, wherein the nanoscale primary particles are selected from oxides of the following elements: silicon, tin, titanium, zirconium, calcium, strontium, barium, aluminium, yttrium, zinc, tantalum, cerium, gadolinium, holmium, erbium, and ytterbium and glass, glass ceramics and ceramic-forming materials and/or combinations thereof.

7. Agglomerate particles according to claim 1, wherein the nanoscale primary particles are functionalised in their surface.

8. Agglomerate particles according to claim 7,
wherein the surface is modified with silanes, silanzanes, siloxanes, carboxylic acids, alcohols, amines, amino acids, sulphonates, phosphates, polyphosphates, ampholytes, thermoplastic homopolymers, copolymers, block copolymers, and/or combinations thereof.

9. Agglomerate particles according to claim 1, wherein the agglomerate particles are hollow or compact.

10. A method for incorporating agglomerate particles in a polymer matrix,
comprising combining agglomerate particles with a particle diameter of 0.5 to 50 μm which are formed by agglomeration of nanoscale primary particles with a particle diameter of 0.5 to 100 nm with the polymer to produce a polymer matrix.

11. The method according to claim 10,
wherein the agglomerate particles have a diameter of 1 to 20 μm.

12. The method according to claim 10
wherein up to 40% by weight, of agglomerate particles, relative to the total mass of the polymer matrix are incorporated.

13. The method according to claim 10,
wherein the agglomerate particles or the agglomerates thereof are present in a master batch.

14. The method according to claim 10,
wherein the agglomerate particles are chosen such that they disintegrate by means of shear forces into the nanoscale primary particles during incorporation in the polymer matrix or a polymer precursor.

15. The method according to claim 14,
wherein, in addition, an energy supply is effected.

16. The method according to claim 10,
wherein the incorporation of the agglomerate particles is effected by means of mixers, kneaders, dissolvers or by means of extrusion in an extruder.

17. The method according to claim 14,
wherein the agglomerate particles are formed by agglomeration of surface-functionalised, nanoscale primary particles.

18. The method according to claim 10,
wherein the agglomeration of the primary particles into agglomerate particles is effected by spray drying, spray freeze drying, freeze drying and/or aerosol methods.

19. The method according to claim 10,
wherein the polymer of the polymer matrix is selected from polyamides, polyurethanes, polyesters which can be saturated or unsaturated, polycarbonates, polyacrilonitriles, butadiene- or isoprene polymers, polystyrenes, polypropylene, polyethylene, polyvinylchloride, polyacrylates, polymethacrylates, polyethers, silicones, inorganic-organic hybrid polymers (ORMOCERs), non-shrinking or expanding polymers, electrically conductive polymers, aminoplasts, melamine resins, polysulphones, polyimides, combinations thereof to form copolymers, blends or, derived therefrom, composites and solvent- or reactive monomer-containing systems which can optionally contain fillers and additives.

20. The method according to claim 10,
wherein the agglomerate particles are spherical.

21. The method according to claim 10,
wherein the agglomerate particles are hollow or compact.

22. The method according to claim 10,
wherein the agglomerate particles are present in a flowable, non-dusty form.

23-26. (canceled)

27. A composition, which is a nanodispersion, nanocomposite, or nanocoating, comprising the agglomerate particles of claim 1.

28. The composition of claim 27, which is a nanodispersion, wherein said nanodispersion is a cosmetic, pharmaceutical or medical aid.

29. The composition of claim 27, which is a nanocomposite, wherein said nanocomposite is a dielectric, electrooptical, electrical, or medical nanocomposite.

30. The composition of claim 27, which is a nanocoating, wherein said nanocoating is a varnish, pain, ink, coating system, flame retardant system or electrorheological fluid.
